(19) United States
(12) Patent Application Publication
(10) Pub. No.: US 2025/0367037 A1
(43) Pub. Date: Dec. 4, 2025

(54) COMPRESSION TROUSERS

(71) Applicant: Julius Zorn GmbH, Aichach (DE)

(72) Inventors: Christian TAEGER, Munich (DE); Mathias LEITLOFF, Bindlach (DE)

(73) Assignee: Julius Zorn GmbH, Aichach (DE)

(21) Appl. No.: 19/218,853

(22) Filed: May 27, 2025

(30) Foreign Application Priority Data
May 29, 2024 (DE) 10 2024 115 125.0

(51) Int. Cl. A61F 13/08

(52) U.S. Cl. CPC A61F 13/08

(57) ABSTRACT

Compression trousers includes a waistband with a first fastening element, a pants part, and a compression element with a second fastening element. The waistband is adjustable by detachable fasteners in a waist or upper hip region of the patient. When the compression trousers are put on the patient, the first fastening element lies in or below an abdominal region, and the pants part fully encloses at least the pelvic region to exert a compressive pressure thereon. The compression element exerts compressive pressure on the genital region. The compression element is configured to be fastened to the waistband by a releasable coupling of the first and second fastening elements. The waistband and the pants part are separable parts or can be separated from each other, and the compression element is designed as a one-piece pouch or apron made of an elastic textile material and is fastened to the pants part.

### Field of the Invention

The invention relates to compression trousers, in particular for a compressive treatment of edema or other diseases or injuries in the genital or pelvic region of a patient, or for supporting postpartum recovery after childbirth.

### BACKGROUND OF THE INVENTION

EP 2 884 862 B1 discloses a medical garment in the form of a hip and abdominal support for supporting the pelvis and abdomen of a patient to treat and control pain, edema and infection associated with acute or chronic conditions such as cancer, or following genital or abdominal surgery or vaginal delivery to reduce complications and improve recovery time. The hip and abdominal support comprises a waist encircling member which can be adjustably fastened at its front portion and has an upper and a lower edge and a rear portion, and the support is further comprising shorts which are non-removably fastened to the lower edge of the waist encircling member and have a crotch portion, and a strap member which is attached at one end to the rear part of the waist-enclosing member and the other end of which, after being passed under the crotch portion of the shorts, can be fixed to the other end of the front side of the waist-enclosing member by means of a hook-and-loop fastener, so that the strap member exerts a compressive pressure on the crotch portion of the wearer, wherein the strap member consists of three straps so that the wearer can attach the other end of one of the three straps to a central part of the front of the waist encircling member and attach the other ends of the remaining two straps laterally displaced with respect to the other end of one of the straps.

The known hip and abdominal support is designed as a one-piece medical garment which is individually adjustable garment and can be worn over the underwear immediately after an operation or after childbirth and afterwards during a recovery period of several weeks of the person being treated, in order to support the back, the abdominal muscles and the vulva. However, the one-piece design of the medical garment makes it difficult to put it on and to take it off. Furthermore, the known garment proves to be disadvantageous in terms of flexibility and an individual adjustment to different patients with different proportions and shapes in the abdominal and hip area. Further disadvantages result from the one-piece design of the medical garment in terms of wearing comfort, e.g. when the patient is bending over, because the element surrounding the waist then is pulled downwards.

In other applications for medical hip and abdominal supports, such as the compressive treatment of genital edema or other edema in the pelvic or groin area, it would also be advantageous when a predetermined compression pressure could be generated in the hip area. The known hip and abdominal support is not suitable for this purpose. In addition, the known hip and abdominal support proves to be uncomfortable if an increased compression pressure on the genital area is to be set with the three straps of the strap element.

### SUMMARY OF THE INVENTION

Against this background, an object of the invention is to provide compression trousers for the compressive treatment of edema or other diseases or injuries in the genital or pelvic area of a patient, which allow an individual and a precise adjustment to the body shape of the patient, the setting of a desired, individually and precisely adjustable compression pressure both in the genital area and the groin area as well as in the hip area, and a comfortable wearing over a longer treatment period. The compression pressure generated by the compression trousers should be easily adjustable during the treatment period with a precise adjustment of the desired compression pressure and the compression pressure should be adaptable to the success of the treatment. Furthermore, the compression trousers should be easy to put on and take off.

The compression trousers are used in particular for the compressive treatment of edema or other diseases or injuries in the genital or pelvic area of a patient and can be worn in particular by male and also by female patients, for example for postpartum recovery after childbirth. The compression trousers comprise a waistband with first fastening means, a pants part and a compression element equipped with second fastening means, wherein the compression element is configured to exert a compression pressure on the genital region of the patient, wherein the waistband can be adjusted by means of detachable fastening elements in the region of the waist or in the upper hip region of the patient, wherein the first fastening means come to lie at least in or below the abdominal region of the patient and the pants part fully encloses at least the pelvic region of the patient in the tightened state, and the compression element is configured to be fastened to the waistband by means of a releasable coupling of the first fastening means and the second fastening means. The waistband and the pants part are separate or separable parts of the compression trousers and the compression element is designed as a one-piece pouch or as a one-piece apron made of an elastic textile material and is permanently attached to the pants part.

The separate or separable design of the waistband and the pants part creates a two-piece compression trousers that are easier and more comfortable to put on a patient and to take off the patient, as compared to a one-piece compression pant. The detachable coupling of the first fastening means and the second fastening means for fastening the compression element to the waistband enables a targeted and precise adjustment of a compression pressure to be exerted by the compression element on the genital area or the groin area of the patient. The first fastening means and the second fastening means are suitably designed such that they can be coupled to one another at different fixing points, wherein the compression pressure exerted by the compression element on the on the genital area or the groin area of the patient is depending on the fixing position, at which the first fastening means and the second fastening means are coupled to one another.

The first fastening means and the second fastening means are preferably designed as corresponding components of a two-part, reversible connecting means. In particular, the first fastening means and the second fastening means are each a part of a Velcro® (hook and loop) fastener, an adhesive fastener or a snap fastener. The first fastening means can be designed, for example, as a flat Velcro® pad, which is arranged on an outer side of the waistband. The second fastening means can be designed as a corresponding Velcro® strip and arranged on a (free) end of the compression element. This allows the second fastening means (Velcro® strip) to be fixed at any position on the Velcro® pad of the first fastening means in order to set a desired compression pressure of the compression element. The higher the fixing position of the Velcro® strip of the second fastening means (Velcro® strip) on the Velcro® pad of the first fastening means, the more the elastic compression element is stretched and the higher the compression pressure exerted by the compression element on the genital area and the groin area of the patient.

The first fastening means are designed preferably flat and in particular in the form of a flat fleece part of a hook-and-loop fastener. The first fastening means are arranged on an outer side of a front section of the waistband, which comes to lie in the abdominal region of the patient, when the waistband is applied to the patient. This enables a large number of possible fixing positions of the Velcro® strap of the second fastening means (Velcro® strap) on the fleece part (Velcro® pad) of the first fastening means in the abdominal region of the patient. This allows a high degree of variation in the setting of a desired compression pressure to be exerted by the compression element on the genital region of the patient.

The waistband can be fixed in the area of the waist or the upper hip area of the patient by means of the detachable fastening elements arranged on the waistband. The detachable fastening elements of the waistband are preferably designed as a Velcro® fastener or a belt buckle, and are arranged in an end section of the waistband. This allows the waistband to be comfortably and securely fastened around the patient's waist or hip circumference, while the waistband is slightly stretched. The fastening elements are preferably arranged in the patient's abdominal area, allowing the patient to comfortably put on and secure the waistband in a fixing position. By fixing the detachable fastening elements at different positions in a circumferential direction of the patient, a desired pressure exerted by the waistband on the waist area and/or the hip area of the patient can be adjusted.

By forming the compression element as a one-piece pouch or as a one-piece apron made of an elastic textile material, a uniform, two-dimensional compression pressure is generated, when the compression element is attached to the waistband by means of a coupling of the first and second fastening means at a selected coupling position, when the compression trousers are put on the patient. In the selected coupling position, the compression element is stretched and thus is exerting a compression pressure on the patient in the genital region. The compression element, which is shaped as a pouch or apron, is preferably shaped and dimensioned anatomically so that the compression element at least largely encloses the patient's genital area.

Due to the bag-shaped or apron-shaped design of the compression element, it has an anatomical fit that can fully encompass the edema in the patient's genital area and thus exerts a uniform, extensive compression pressure on the edema.

In one embodiment of the compression trousers for men, the pouch-shaped compression element is adapted to the patient's anatomy in such a way that the genitals can be completely absorbed by the compression element. This allows the compression element to fully surround the edema to be treated. The bag-shaped compression element in this embodiment also acts as a support bag for the genitals.

The compression trousers according to the invention therefore enable an overall individual and precise adjustment of a compression pressure exerted by the compression trousers on a genital edema or on the lower abdomen and the hip area of the patient, as well as an improved wearing comfort. The waistband, which can be put on separately from the pants part, makes it easier to put on and take off the compression trousers and allows an increased flexibility in the height adjustment for different body circumferences of a patient and enables an efficient pressure adjustment on the lower abdomen, in order to treat an edema effectively. The flat first fastening means on the front of the waistband, which are positioned in the patient's abdominal area when the waistband is in place, allow the compression pressure to be precisely adjusted to the patient's genital and groin area. An edema in the genital or groin area of a patient thereby can be completely enclosed by the compression element, which is designed as a one-piece bag or as a one-piece apron made of an elastic textile material, in order to exert a uniform and individually adjustable compression pressure on the edema. The flat design of the compression element prevents it from cutting into the patient's sensitive soft tissue, which improves the wearing comfort.

A first end of the compression element is attached to the pants part, for example with a non-detachable connection, in particular with a seam. The non-detachable connection is arranged in particular on an inner side of the pants part in a crotch area, which comes to lie in the crotch of the patient when the pants part is put on. In an alternative embodiment, the compression element can also be attached to the inside of the pants part with a detachable connection, e.g. a Velcro® fastener or by means of press studs. The detachable connection is also located in the inner crotch area of the pants part. A detachable connection of the compression element to the pants part has the advantage that the compression element can be replaced. This can be advantageous, for example, if a different pressure range is to be generated by the compression element during a treatment phase of the compression therapy. For example, a first compression element with a defined stretchability can then be replaced by a second compression element with a different stretchability.

At a second end of the compression element, which is opposite the first end, the second fastening means, which can in particular be designed as a Velcro® strip, are arranged. Preferably, the second fastening means are in the form of a hook and loop tape with barbs. This enables the coupling of the first and the second fastening means to be fixed to each other more easily and quickly in a suitable fixing position, as well as an adjustable stretching and thus an adjustable compression pressure of the compression element, when the compression trousers are put on a patient.

When the compression trousers are put on a patient, the compression element, which is formed in particular from an elastic textile material, is detachably fixed to the waistband with a stretching degree that depends on the position of the coupling of the first and the second fastening means. Due to its stretching, the compression element exerts a compression pressure on the genital area and the groin area of the patient, wherein the strength of the compression pressure is depending on the degree of the stretching of the elastic material of the compression element. The compression pressure thus can be adjusted by selecting a coupling position, at which the second fastening means are coupled to the first fastening means. The compression pressure exerted by the compression element can be changed even after the compression trousers have been put on the patient, for example to adjust the compression pressure over the course of a longer treatment period. To adjust the compression pressure, it is only necessary to change the coupling position of the first and the second fastening means without having to remove the entire compression trousers.

Advantageous with regard to wearing comfort of the compression trousers is a partial overlap of an upper section of the pants part, in particular a waistband section of the pants part, with the waistband arranged underneath, when the compression trousers are put on the patient. However, it is possible for the pants part to be positioned on the patient completely below and at a distance from the waistband.

In a preferred embodiment, in which a waistband region of the pants part overlaps with the waistband at least in some areas, second fastening elements, in particular in the form of a plurality of hook parts of a hook-and-loop fastener, are arranged on an inner side of the pants part, in particular in the waistband region. With these second fastening elements the pants part can be detachably fastened to an outer side of the underlying waistband. This results in a two-part embodiment of the compression trousers, in which the pants part and the waistband are designed as separable parts, since the connection of the pants part and the waistband can be separated from each other via the detachably designed second fastening elements. This makes it easier to take off and put on the compression trousers. By “separable” it is meant that a separation of the pants part from the waistband is enabled by releasing them from each other manually without aids, in particular by releasing the second fastening elements with which the pants part is fastened to the waistband.

To improve the wearing comfort of the compression trousers, two lateral longitudinal edges of the compression element can be turned inwards at least partially and in particular in an upper section of the compression element. This also improves the full enclosure of an edema by the compression element. Furthermore, to improve the fit of the compression trousers on the patient, suspenders can be arranged on the pants part and/or on the waistband. For a stable fixing of the compression element in a desired wearing position in the genital area of the patient, additionaly fastening straps can be arranged on the compression element, in particular in the area of the lateral longitudinal edges of the compression element.

The pants part of the compression trousers is preferably a compression textile made from an elastic textile material and is made in particular from an elastic compression knit, the textile material preferably being selected and the pants part being shaped and dimensioned in such a way that the pants part exerts a predetermined compression pressure on the patient, in particular in the hip and pelvic region. The pants part is preferably made from an elastic compression knit with a base knit and an elastic weft thread inserted therein. The pants part in particular can be made from a circular knitted fabric. This makes it possible to set the desired compression pressure by selecting and stretching the weft yarn material and adapting the dimensions of the pants part to the patient's anatomy, in particular by individually making it to measure. By designing the pants part as a compression textile, in addition to the pressure exerted by the compression element on the patient's genital area, a fully effective compression pressure is also generated on the patient's hip area and/or pelvic area. This means that an edema extending from the genital area via the groin area to the hip and pelvic area can be effectively treated with the compression trousers by a compression therapy.

Both the pants part and the compression element are conveniently each made from an elastic textile material. Preferably, the pants part is made of a compression knit or an elastic woven textile material. The compression element can also be made of a compression knit or a woven textile material. The waistband is preferably made of a foam laminated on both sides with a velour material. The elasticity of the waistband is preferably less than the elasticity of the pants part and the compression element.

When the compression trousers are put on the patient, the compression element is preferably located inside the pants part. The compression element is conveniently and at least substantially completely covered by the pants part, except for an upper edge section at the free end (second end) of the compression element, which may protrude from the pants part, in particular from the waistband of the pants part. The protrusion of an upper edge section at the free end of the compression element from the pants part is advantageous because it allows the protruding free end of the compression element to be gripped manually, for example in order to be able to adjust or readjust the compression pressure exerted by the compression element by fixing the coupling of the first and the second fastening means at a different coupling position.

An arrangement of the compression element inside the pants part is advantageous in terms of an improved wearing comfort because, for example, pressure points which can be caused by a fold of a pants part lying under the compression element, can be avoided. In addition, the compression element can be fixed in the desired position in the patient's genital area by the pants part, which is elastic in particular, when the compression element is inside the pants part. Preferably, the compression element arranged inside the pants part is completely enclosed by the pants part, so that the compression element is completely covered by the pants part. A compression element completely concealed by the pants part is also advantageous for aesthetic reasons.

In order to arrange the compression element inside the pants part, it is useful if a first end of the compression element is fastened to an inner side of the pants part and the second fastening means are arranged at a free, second end of the compression element. This ensures that the compression element is located inside the pants part and enables the second end of the compression element to be detachably fixed to the waistband by means of the coupling of the first fastening means and the second fastening means when the compression trousers are put on the patient. The compression pressure exerted by the compression element can be set to desired values via the selectable fixing position of the fixation of the second fastening means on the waistband.

In a preferred embodiment, a pad is provided on the inside of the compression element, which faces the patient's body when the compression trousers are put on. Preferably, the pad can be inserted into the compression element and detachably attached to the compression element, for example via a Velcro® fastener. The pad serves to increase the pressure exerted by the compression element on an edema and has an appropriate shape adapted to the body shape of the patient to be treated. In particular, pads with a fit suitable for men or women can be used in the crotch or perineal area. In addition to or instead of fastening the pad to the inside of the compression element by means of a detachable fastening means, such as a Velcro® fastener, a pocket can also be provided on the compression element, in particular on its inside, into which the pad can be removably inserted.

### BRIEF DESCRIPTION OF THE DRAWINGS

These and other advantages and preferred features of the invention are apparent from the embodiment example described below with reference to the drawings. The drawings show

FIG. 1: Front view of compression trousers with a waistband and a pants part with an integrated compression element in a state applied to a patient;

FIG. 2: Rear view of the compression trousers of FIG. 1 in a state applied to the patient;

FIG. 3: Front view of the compression trousers of FIG. 1 with the pants part pulled down to better show the compression element and the waistband of the compression trousers;

FIG. 4: Rear view of the compression trousers of FIG. 3 with the pants part pulled down;

FIG. 5: Front view of the compression trousers of FIG. 1 with the pants part pulled down and the compression element unfolded to better show the inside of the compression element and the waistband of the compression trousers;

FIG. 6: Detailed view of the inside of the pants part of the compression trousers with the compression element attached to it in the crotch area of the pants part, wherein the inside of the compression element is shown;

FIG. 7: Detailed view of the inside of the pants part of the compression trousers of FIG. 1 with the compression element attached to it in the crotch area of the pants part, wherein the outside of the compression element is shown;

FIG. 8: Detailed rear view of the pants part of the compression trousers of FIG. 1 with the compression element attached to it in the crotch area of the pants part; and

FIG. 9: Detailed view of the area shown as a dotted line in FIG. 8, which shows the inside of the compression element.

### DETAILED DESCRIPTION

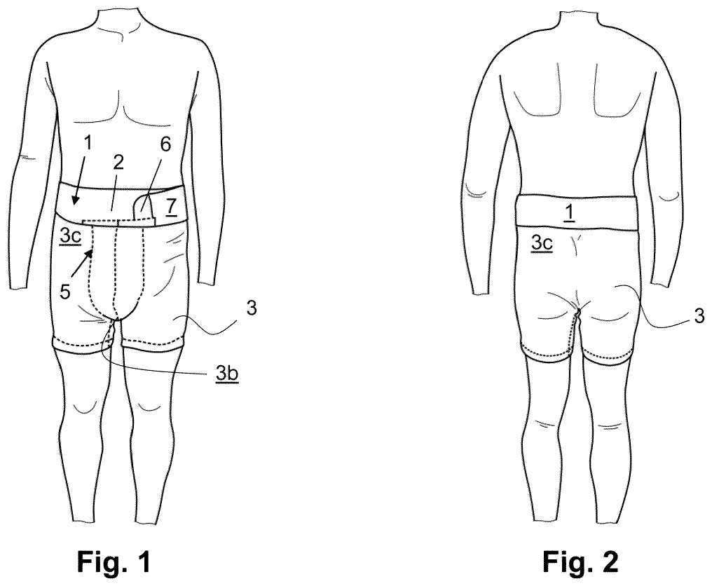

The compression trousers shown in FIGS. 1 and 2 in a front view and a rear view in a state when worn on a patient, can be used in particular for the compressive treatment of oedema or other diseases or injuries in the genital or pelvic region of a patient. The compression trousers comprise a waistband 1, a pants part 3 separated therefrom and a compression element 5 attached to the pants part 3 and arranged on the inside of the pants part 3, which is shown in dashed lines in the illustration of FIG. 1, as it is covered by the pants part 3.

The waistband 1, which can be seen in detail in particular in the illustration in FIG. 5, is designed as a band which fully surrounds the patient's waist when applied to the patient and contains a central section 1a which is extended downwards (i.e. in the distal direction) compared to the edge sections adjoining the central section 1a on both sides. The central section 1a, which comes to lie in the abdominal region of the patient when the waistband 1 is applied to the patient, is designed as a flat fleece part of a hook-and-loop fastener and thus forms the first fastening means 2.

At one end 7 of the waistband 1, detachable fastening elements 6 are arranged. These fastening elements 6 can be, for example, a Velcro® element with a hook of a Velcro® fastener. The fastening elements 6 can also be a belt buckle, for example. The waistband 1 can be fixed in the area of the patient's waist by means of the detachable fastening elements 6 by coupling the fastening elements 6, for example by fastening a Velcro® fastener or by closing a belt buckle. By positioning the fixation point of the fastening elements 6, a selected pressure exerted by the waistband 1 on the patient's waist can be adjusted. To remove the waistband 1 from the patient, the fastening elements 6 can be released, for example by releasing the coupling of the Velcro® fastener or opening the belt buckle.

Below the waistband 1, the pants part 3 of the compression trousers is located in partial overlap with the underlying waistband 1 when the compression trousers are put on the patient. In the embodiment example shown in the drawing, the pants part 3 is designed as a short with a tubular torso section 3a, which contains a crotch area 3b in the middle, two tubular leg sections 3c arranged on the torso section 3a and a waistband 3d arranged on the upper edge of the torso section 3a. The length of the two leg sections 3c is variable, i.e. the length of the leg sections 3c can be longer or shorter than in the embodiment example shown here in the drawing. In particular, the pants part 3 can also be designed as tights with leg sections 3c, whereby the leg sections can extend up to the patient's ankle and can optionally also include a foot section.

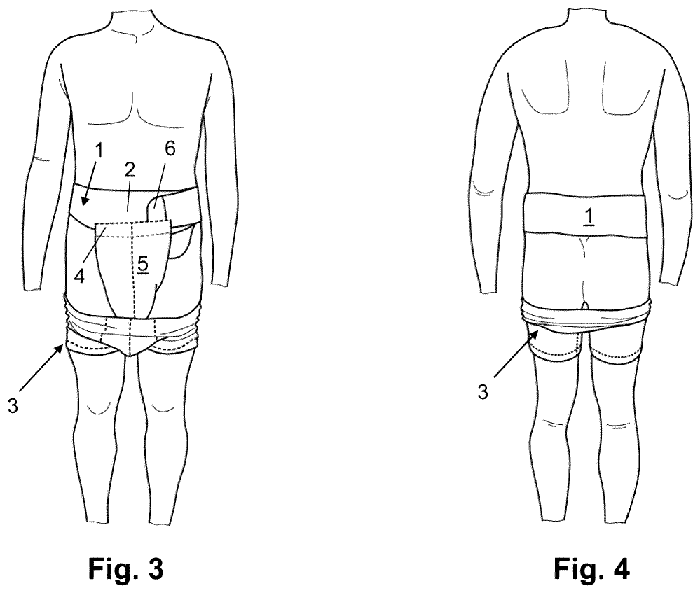

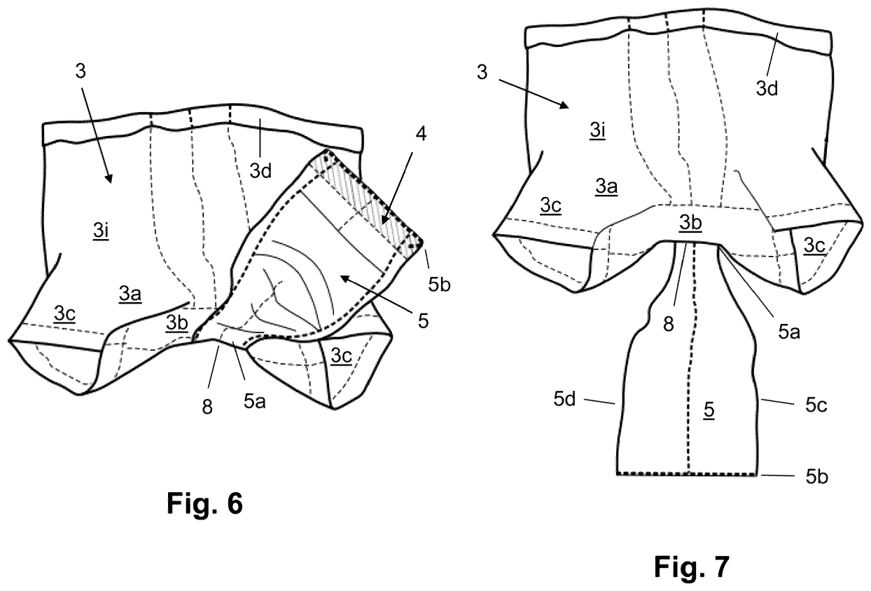

A compression element 5 is attached to the inside 3i of the pants part 3, which can be seen in FIGS. 6 and 7, in the crotch area 3b. The compression element 5, which can be seen in particular in FIGS. 3, 5 and 6 to 9, is formed as a one-piece pouch or as a one-piece apron made of an elastic textile material. The compression element 5 has a first end 5a and an opposite second end 5b and lateral longitudinal edges 5c, 5d extending there between, as shown in FIG. 7. The first end 5a of the compression element 5 is attached to the inside 3i of the pants part 3 in the crotch area 3b by means of a seam 8, as shown in FIGS. 5 to 9. The second fastening means 4 are arranged at the free second end 5b of the compression element 5. In the embodiment example shown in the drawing, the second fastening means 4 is a Velcro® strip, which is arranged in the region of the upper edge at the free second end 5b of the compression element 5. The second fastening means 4 serve to detachably fasten the compression element 5 to the first fastening means 2, which are provided on the front central section 1a of the waistband 1. Preferably, the second fastening means 4 are in the form of a hook-and-loop strip with hooks, which interact with a hook-and-loop pad (loop part) of a hook-and-loop fastener (Velcro® fastener) arranged in the front central section 1a of the waistband 1. By coupling the second fastening means 4 of the compression element 5 with the first fastening means 2 of the waistband 1, the free second end 5b of the compression element 5 can be detachably fixed at a selected fixing position on the waistband 1. The fixing of the free second end 5b of the compression element 5 to the front central section 1a of the waistband 1 can be seen, for example, in the illustration in FIG. 3. In FIG. 5, the compression element 5 is shown in a state in which it has been removed from the waistband 1, wherein the inside of the compression element 5, on which the second fastening means 4 are arranged in the region of the free second end 5b, can be seen.

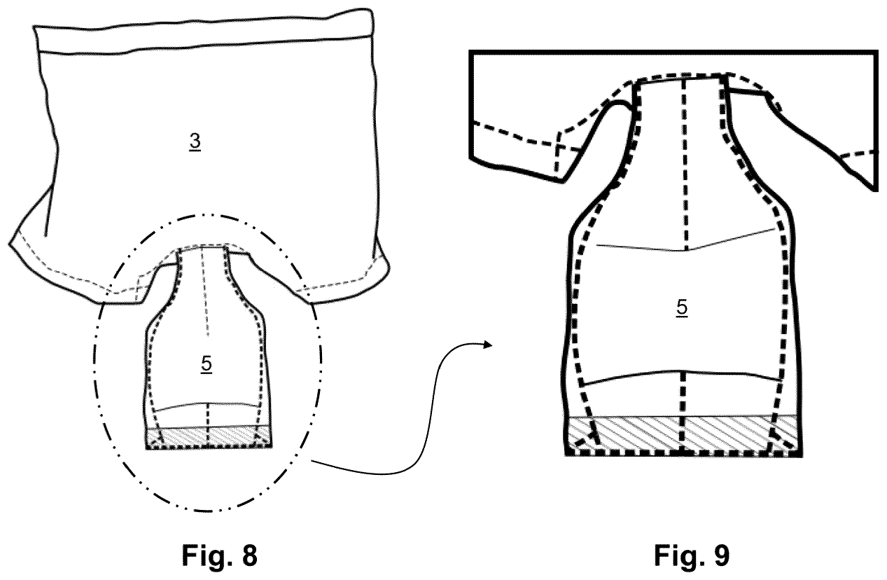

The inside of the compression element 5 with the second fastening means 4 arranged there at the free second end 5b can also be seen in FIG. 6. FIG. 7 shows the outside of the compression element 5 with the two lateral longitudinal edges 5c, 5d. FIGS. 8 and 9 show a detailed view of the inside of the compression element 5. FIGS. 8 and 9 show that the two lateral longitudinal edges 5c, 5d of the compression element 5 are turned inwards, i.e. towards the inside, in a section in the region of the free second end 5b. The folded-in lateral longitudinal edges are fixed with a seam and are shown as dashed lines in FIGS. 8 and 9.

To put the compression trousers on a patient, the waistband 1 is first placed around the patient's waist and fixed in place using the detachable fastening elements 6. By selecting the fixing position of the detachable fastening elements 6, a certain amount of pressure can be applied and is exerted by the waistband 1 on the patient's waist and hip area.

After putting on the waistband 1, the patient puts on the pants part 3, whereby the compression element 5 sewn on in the crotch area 3b of the pants part 3 comes to rest on the inside 3i of the pants part 3. The free second end 5b of the compression element 5 is then pulled out of the pants part 3 at the top of the pants part 3, in particular in the area of the waistband 3d of the pants part 3, as indicated in FIG. 1 by the dotted lines of the compression element 5. Except for the upper edge section at the free end (second end 5b) of the compression element 5, which is shown in dotted lines in FIG. 1 and protrudes from the waistband 3d of the pants part 3, the compression element 5 is located inside the pants part 3 when the compression trousers are put on the patient. The compression element 5 conveniently is substantially covered by the pants part 3, except for an edge section of the compression element 5 protruding from the waistband 3d of the pants part 3.

By means of the second fastening means 4, which are arranged on the inside of the compression element 5 in the area of the free second end 5b, the compression element 5 is fixed to the waistband 1 by coupling the second fastening means 4 with the first fastening means 2. Due to the elastic properties of the compression element 5, which is formed from an elastic textile material, the compression element 5 is stretched more or less depending on the fixing position of the first and the second fastening means 2, 4. The degree of stretching of the compression element 5 can be used to select a compression pressure exerted by the compression element 5 on the genital area and the groin area of the patient. The fixing position, at which the second fastening means are coupled to the flat first fastening means, thus determines the compression pressure exerted by the compression element 5 on the patient's genital area.

In a preferred embodiment, second fastening elements 9, in particular in the form of a plurality of hook parts of a hook and loop fastener or press studs, are provided on the inside 3i of the pants part 3 in the area of its waistband 3d. With these second fastening elements 9, the inner side 3i of the pants part 3, in particular in the area of the waistband 3d of the pants part 3, can be removably fastened to the outer side of the waistband 1 lying underneath in an overlapping area. If the second fastening elements 9 are press studs, for example, the outside of the waistband contains a plurality of corresponding receptacles with recesses corresponding to the number of press studs, into each of which a head part of a press stud arranged on the inside of the waistband 3d of the pants part 3 can be releasably anchored, or vice versa.

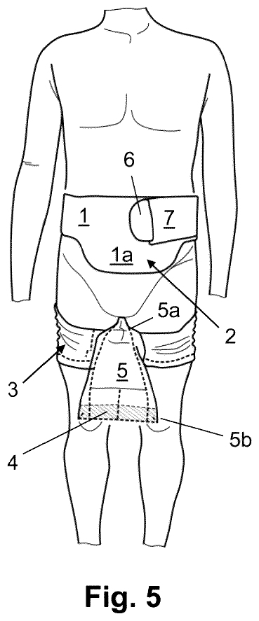

To remove the compression trousers from the patient, the procedure described above for putting the compression trousers on a patient is carried out in reverse order. For this purpose, the pants part 3 is first pushed downwards, as shown in FIG. 3 and FIG. 4, and then the coupling of the second fastening means 4 to the first fastening means 2 can be released, in order to release the connection of the free second end 5b of the compression element 5 to the waistband 1. The pants part 3 with the integrated compression element 5 can then be removed and finally the waistband 1 can be removed from the patient's body.

The two-piece design of the disclosed compression trousers with the waistband 1 and the separate or separable pants part 3 with the integrated compression element 5 makes it easy and convenient to put on and take off the compression trousers. When putting on the compression trousers, a certain stretch of the elastic compression element 5 can be set by adjusting the coupling position at which the second fastening means 4 are coupled to the flat first fastening means 2. By adjusting the stretching of the compression element 5, the compression pressure exerted by the compression element 5 on the genital region and the groin area of the patient can be selected and adjusted. The compression pressure exerted by the compression element 5 can also be subsequently re-adjusted, i.e. after the compression trousers have been put on the patient. This is advantageous, for example, in the case of a longer-lasting compression therapy, in order to be able to adapt the compression pressure to the healing process. For example, it is possible to gradually reduce the compression pressure during the course of a treatment period, depending on the healing process.

Both the pants part 3 and the compression element 5 are each appropriately made from an elastic textile material. Preferably, the pants part 3 is made of a compression knit with a basic knit of an elastic thread and an elastic weft thread inserted or knitted into it, or a circular knitted fabric. In particular, the material of the pants part 3 can be a circular knitted fabric made of 80% polyamide and 20% elastane. The compression element 5 can also be formed from a compression knit. The compression element 5 is preferably a woven textile material, for example made of 71% polyamide and 31% elastane. The waistband 1 is preferably formed from a foam material laminated on both sides with a velour material, for example 74% polyamide, 11% polyurethane and 15% elastane. The stretchability of the waistband 1 is preferably lower than the stretchability of the pants part 3 and the compression element 5.

1. Compression trousers for a compressive treatment of edema or other diseases or injuries in the genital or pelvic region of a patient, the compression trousers comprising:
a waistband equipped with first fastening means,
a pants part, and
a compression element equipped with second fastening means,
wherein the waistband is adjustable by means of detachable fastening elements in a waist region or in an upper hip region of the patient,
wherein, when the compression trousers are donned to the patient, the first fastening means are lying at least in or below an abdominal region of the patient, and the pants part fully encloses at least the pelvic region of the patient to exert a compressive pressure thereon, and the compression element is exerting a compressive pressure on the genital region of the patient,
wherein the compression element is configured to be fastened to the waistband by means of a releasable coupling of the first fastening means and the second fastening means,
wherein the waistband and the pants part are separable parts of the compression trousers or can be separated from each other, and the compression element is designed as a one-piece pouch or as a one-piece apron made of an elastic textile material and is fastened to the pants part.

2. The compression trousers according to claim 1, wherein the compression element at least substantially encloses the genital region of the patient, when the compression trousers are donned to the patient.

3. The compression trousers according to claim 1, wherein the compression element is fastened to the pants part with a connection, wherein the connection is arranged in a crotch region, which comes to lie in the crotch of the patient, when the pants part is put on the patient.

4. The compression trousers according to claim 3, wherein the compression element comprises a first end and a second end, wherein the first end is attached to the pants part in the crotch region.

5. The compression trousers according to claim 4, wherein the second fastening means are arranged at the second end of the compression element.

6. The compression trousers according to claim 4, wherein the first end of the compression element is fastened to an inner side of the pants part and the second end of the compression element is detachably fixed to the waistband by means of the coupling of the first fastening means and the second fastening means, when the compression trousers are put on the patient.

7. The compression trousers according to claim 1, wherein the compression element is made of an elastic material and is attached to the waistband with a stretching of the elastic material, when the compression trousers are put on the patient, whereby the compression element exerts a compression pressure on the genital region of the patient, wherein the compression pressure is adjustable by selecting a coupling position at which the second fastening means are coupled to the first fastening means.

8. The compression trousers according to claim 1, wherein the pants part is arranged completely below the waistband or the pants part is at least partially overlapping with the waistband in an overlapping region, wherein the waistband is lying under the pants part in the overlapping region, when the compression trousers are put on the patient.

9. The compression trousers according to claim 8, further comprising second fastening elements arranged on an inner side of the pants part in an upper section of the pants part, wherein the upper section of the pants part overlaps with the waistband arranged therebelow, when the compression trousers are put on the patient, and wherein the pants part can be detachably fastened by means of the second fastening elements on an outer section of the waistband lying below the upper section of the pants part.

10. The compression trousers according to claim 1, wherein the first fastening means are flat and are designed as a flat fleece part of a hook-and-loop fastener, wherein the first fastening means are arranged on an outer side of a front section of the waistband, which is lying in the abdominal region of the patient, when the waistband is put on the patient.

11. The compression trousers according to claim 1, wherein the compression element has two longitudinal edges, wherein each longitudinal edge is turned inwards at least partially.

12. The compression trousers according to claim 1, further comprising suspenders arranged on the pants part or on the waistband.

13. The compression trousers according to claim 1, further comprising fastening straps arranged on the compression element.

14. The compression trousers according to claim 1, wherein the first fastening means and the second fastening means are designed to be mutually corresponding components of a two-part reversible connecting means.

15. The compression trousers according to claim 1, wherein the pants part is designed to be a compression textile made of an elastic textile material, wherein the textile material is stretchable in such a way that the pants part exerts a predetermined compression pressure in the hip and pelvic region of the patient upon stretching of the elastic textile material, when the compression trousers are donned to the patient.

16. The compression trousers according to claim 1, wherein the detachable fastening elements are arranged in an end section of the waistband.

17. The compression trousers according to claim 1, wherein the detachable fastening elements are configured to detachably fasten the waistband to the patient in the patient's abdominal region, wherein the detachable fastening elements are configured to be fixed at different fixing positions in a circumferential direction of the patient, wherein the fixing positions of the detachable fastening elements are defining a degree of a compression pressure exerted by the waistband on the waist region and/or the hip region of the patient.

18. The compression trousers according to claim 1, further comprising a pad which is configured to be detachably attached to the compression element.

19. The compression trousers according to claim 1, wherein the compression element lies inside the pants part when the compression trousers are put on the patient.
